(19) United States
(12) Patent Application Publication
(10) Pub. No.: US 2012/0315684 A1
(43) Pub. Date: Dec. 13, 2012

(54) Plasma Oxidation-Reduction Method, Method for Promoting Plant/Animal Growth Using the Same, and Plasma-Generating Device for Use in Method for Promoting Plant/Animal Growth

(75) Inventors: Nobuya Hayashi, Saga-shi (JP); Akari Nakahigashi, Saga-shi (JP); Masaharu Shiratani, Fukuoka-shi (JP); Kazunori Koga, Fukuoka-shi (JP)

(73) Assignees: Saga University, Saga-shi (JP); Kyushu University, National University Corporation, Fukuoka-shi (JP)

(21) Appl. No.: 13/575,986

(22) PCT Filed: Jan. 31, 2011
(86) PCT No.: PCT/JP2011/051940

§ 371 Date: Jul. 30, 2012

(30) Foreign Application Priority Data
Jan. 31, 2010 (JP) 2010-019839
Nov. 29, 2010 (JP) 2010-265654

(51) Int. Cl. C12N 13/00; A01G 7/04; B01J 19/12

(52) U.S. Cl. 435/173.8; 204/165; 47/58.1 R

(57) ABSTRACT

Provided is a plasma oxidation-reduction method with which it is possible to control the structure of amino acids and proteins with high and stable reproducibility, by using plasma in order to control the amino acids and proteins that make up a living body, particularly by using plasma in order to oxidize or reduce amino acids and proteins. Also provided are a method for promoting plant/animal growth using the plasma oxidation-reduction method, and a plasma-generating device for use in the method for promoting plant/animal growth. Amino acids or proteins are oxidized or reduced in the plasma oxidation-reduction method by using an active oxygen species or active hydrogen in the plasma. Preferably, the active oxygen species comprises any one of singlet oxygen atoms, excited oxygen molecules, or hydroxyl radicals, and the active hydrogen comprises excited hydrogen atoms.

### TECHNICAL FIELD

This invention relates to a plasma oxidation-reduction method, a method for promoting plant/animal growth using the same, and a plasma-generating device for use in the method for promoting plant/animal growth, and in particular to a plasma oxidation-reduction method for oxidizing or reducing amino acids or proteins by using plasma, a method for promoting plant/animal growth using the same, and a plasma-generating device for use in the method for promoting plant/animal growth.

### BACKGROUND ART

In recent years, plasma applications have been expanding rapidly, and in the medical field, for example, plasma is used to sterilize medical instruments so that not only microorganisms, such as bacteria and viruses, but also refractory organic proteins having infectivity are attempted to be decomposed. As an example of this, the present applicants disclosed a method for sterilizing long tubes, such as catheters, in Patent Document 1.

Meanwhile, it has been known that the falling of a thunderbolt may promote the growth of certain kinds of produce (rice, grapes). In recent years, it has been confirmed that pulse voltages can be applied to shiitake mushroom-growing logs in order to increase the yield of the shiitake mushrooms, and thus, the voltage-applying method has been used practically for the cultivation of shiitake mushrooms.

Other methods have also been proposed, for example, a method for promoting the growth or the propagation of a living thing by exposing the living body to negative ions as in Patent Document 2, a method for promoting the growth of a plant by using water having a high ozone concentration that is gained through plasma discharge as in Patent Document 3, a method for promoting the growth of cattle or fish through negative ionization by generating negative ions through corona discharge as in Patent Document 4, and a method for promoting the growth of a plant by ionizing carbonic acid gas using plasma as in Patent Document 5.

However, it is still unclear how the discharge or the plasma contributes to the growth of animals/plants, and these techniques lack reproducibility, and therefore are not sufficiently used at present. In addition, plasma itself has a high energy level and can possibly destroy or decompose amino acids or proteins that make up a living body, and thus, there is a concern that plasma may damage a living body.

### PRIOR ART DOCUMENTS

### Patent Documents

Patent Document 1: Japanese Patent No. 4214213
Patent Document 2: Japanese Unexamined Patent Publication 2006-325493
Patent Document 3: Japanese Unexamined Patent Publication 2006-289236
Patent Document 4: Japanese Unexamined Patent Publication H9 (1997)-172907
Patent Document 5: Japanese Unexamined Patent Publication H3 (1991)-72819

### SUMMARY OF THE INVENTION

### Problem to Be Solved by the Invention

An object to be achieved by the present invention is to solve the above-described problems and to provide a plasma oxidation-reduction method with which it is possible to control the structure of amino acids and proteins with high and stable reproducibility, by using plasma in order to control the amino acids and the proteins that make up a living body, particularly by using plasma in order to oxidize or reduce the amino acids and the proteins. Other objects are to provide a method for promoting plant/animal growth using this plasma oxidation-reduction method, and a plasma-generating device for use in the method for promoting plant/animal growth.

### Means for Solving Problem

In order to achieve the above-described objects, the present invention has the following technical features.

(1) A plasma oxidation-reduction method is characterized in that amino acids or proteins are oxidized or reduced by using an active oxygen species or active hydrogen in plasma.

(2) The plasma oxidation-reduction method according to the above (1) is characterized in that the active oxygen species includes any one of singlet oxygen atoms, excited oxygen molecules or hydroxyl radicals, and the active hydrogen includes excited hydrogen atoms.

(3) The plasma oxidation-reduction method according to the above (1) or (2) is characterized in that the active oxygen species is generated from water vapor plasma or oxygen plasma, and the active hydrogen is generated from water vapor plasma or hydrogen plasma.

(4) The plasma oxidation-reduction method according to any of the above (1) to (3) is characterized in that the active oxygen species or the active hydrogen is generated through high frequency discharge or microwave discharge.

(5) The plasma oxidation-reduction method according to any of the above (1) to (4) is characterized in that water vapor plasma is used as the plasma, and oxidation occurs due to the active oxygen species when the water vapor pressure is in a range from 100 Pa to 150 Pa.

(6) The plasma oxidation-reduction method according to any of the above (1) to (4) is characterized in that water vapor plasma is used as the plasma, and reduction occurs due to the active hydrogen when the water vapor pressure is in a range from 3 Pa to 30 Pa.

(7) The plasma oxidation-reduction method according to any of the above (1) to (6) is characterized in that an active oxygen species or active hydrogen in a plasma diffusion region is used.

(8) The plasma oxidation-reduction method according to any of the above (1) to (7) is characterized in that the amino acids are within cells.

(9) The plasma oxidation-reduction method according to any of the above (1) to (8) is characterized in that the active oxygen species or the active hydrogen directly oxidizes or reduces the amino acids or the proteins.

(10) The plasma oxidation-reduction method according to any of the above (1) to (8) is characterized in that there is moisture around the amino acids or the proteins, the active oxygen species converts the moisture into hydrogen peroxide, and the hydrogen peroxide oxidizes the amino acids or the proteins.

(11) The plasma oxidation-reduction method according to any of the above (1) to (10) is characterized in that the amino acids are within transcription factors.

(12) The plasma oxidation-reduction method according to any of the above (1) to (11) is characterized in that the amino acids are cystein or cystine.

(13) The plasma oxidation-reduction method according to the above (12) is characterized in that the active oxygen species oxidizes cystein.

(14) The plasma oxidation-reduction method according to the above (12) is characterized in that the active hydrogen reduces cystine.

(15) The plasma oxidation-reduction method according to the above (8) is characterized in that an object with cells having moisture is irradiated with the active oxygen species so that the moisture is converted to hydrogen peroxide and the hydrogen peroxide oxidizes cystein in the transcription factors within the cells, and the transcription factors are activated.

(16) The plasma oxidation-reduction method according to the above (8) is characterized in that an object with cells having moisture is irradiated with the active oxygen species so that the moisture is converted to hydrogen peroxide and the hydrogen peroxide oxidatively modifies cystein within the cells so as to generate cysteic acids, and the cysteic acids activate the transcription factors within the cells.

(17) A method for promoting plant/animal growth is provided using the plasma oxidation-reduction method according to any of the above (1) to (16).

(18) The method for promoting plant/animal growth according to the above (17) is characterized in that an object irradiated with the active oxygen species or the active hydrogen is plant or animal cells.

(19) The method for promoting plant/animal growth according to the above (18) is characterized in that the plant or animal cells are of a seed, the plant or animal cells are contained in a vacuum container, and the plant or animal cells are irradiated with an active oxygen species or active hydrogen through high frequency discharge or microwave discharge.

(20) The method for promoting plant/animal growth according to the above (18) is characterized in that the plant or animal cells have a size of 5 mm or less, and the plant or animal cells are placed in the air at a distance of 1 mm to 15 mm away from the electrode for dielectric barrier discharge, and the plant or animal cells are irradiated with the active oxygen species or the active hydrogen.

(21) The method for promoting plant/animal growth according to the above (20) is characterized in that the plant or animal cells are dispersed in a liquid.

(22) A plasma-generating device for use in a method for promoting plant/animal growth is characterized by having: a vacuum container; an inductively coupled antennae placed in the proximity of a wall of the container; and a means for placing plant or animal cells in a region where the plasma generated by the antennae diffuses.

(23) A plasma-generating device for use in a method for promoting plant/animal growth is characterized by having a pair of electrodes for dielectric barrier discharge, each of which is made of a number of metal rods coated with a ceramic tube, that are meshed with each other so that the metal rods are parallel to each other, wherein the electrodes and plant or animal cells are placed in the air at a distance of 1 mm to 15 mm away from each other.

### Effects of the Invention

According to the present invention, amino acids or proteins are oxidized or reduced using an active oxygen species or active hydrogen in plasma, and therefore, it is possible to control the structure of amino acids or proteins with high and stable reproducibility. In particular, the active oxygen species oxidizes cystein, which is an amino acid within cells, or the active hydrogen reduces cystine so that the degree of activation of the transcription factors can be controlled. Thus, such controls are also possible that the growth of a living body is promoted or inhibited. Furthermore, it is possible to provide a method for promoting plant/animal growth by using this plasma oxidation-reduction method. The use of the plasma-generating device according to the present invention also makes it possible to implement the method for promoting plant/animal growth in a vacuum container or in the air.

### BRIEF DESCRIPTION OF THE DRAWINGS

FIGS. 1A and 1B are diagrams showing an example of a plasma-generating device for use in the plasma oxidation-reduction method (method for promoting plant/animal growth) according to the present invention;

FIG. 2 is a graph showing a change in the amount of active hydrogen or hydroxyl radicals relative to the change in the water vapor pressure when water vapor plasma is generated in the plasma-generating device in FIGS. 1A and 1B;

FIG. 3 is a graph showing a change in the FTIR spectrum before and after cystein is oxidatively treated;

FIG. 4 is a graph showing a change in the FTIR spectrum before and after cystine is reductively treated;

FIG. 5 is a graph showing the dependency of the length (stem plus root) of a daikon radish sprout on the period of time irradiated with water vapor plasma;

FIG. 6 is a graph showing the dependency of the amount of thiol in a seed of a daikon radish sprout on the period of time irradiated with water vapor plasma;

FIG. 7 is a graph showing the dependency of the absorbance by the disulfide bond in the FTI spectrum of a cystein sample on the water vapor pressure;

FIG. 8 is a graph showing the dependency of the absorbance by the thiol group in the FTI spectrum of a cystein sample on the water vapor pressure;

FIG. 9 is a diagram showing another example of the plasma-generating apparatus for use in the plasma oxidation-reduction method (method for promoting plant/animal growth) according to the present invention;

FIG. 10 is a diagram schematically showing the circuit structure of the plasma-generating device in FIG. 9;

FIG. 11 is a graph showing the current-voltage waves in the plasma-generating device in FIGS. 9 and 10;

FIG. 12 is a diagram for illustrating the positional relationship between the electrodes and an object in the plasma-generating device;

FIG. 13 is a cross-sectional diagram along arrow Y-Y in FIG. 12;

FIG. 14 is a graph for illustrating the state of budding yeast immediately after being irradiated with air plasma in the atmospheric pressure;

FIG. 15 is a graph for illustrating the state of the budding yeast in FIG. 14 after 38 hours have passed since being irradiated with plasma;

FIG. 16 is a graph showing the tendency according to which the budding yeast increases relative to the period of time irradiated with plasma; and

FIG. 17 is a graph showing the tendency according to which the budding yeast increases relative to the number of times irradiated with plasma.

### BEST MODE FOR CARRYING OUT THE INVENTION

The plasma oxidation-reduction method, the method for promoting plant/animal growth using the same, and the plasma-generating device for use in the method for promoting plant/animal growth according to the present invention are described below in detail.

The plasma oxidation-reduction method according to the present invention is characterized in that amino acids or proteins are oxidized or reduced using an active oxygen species or active hydrogen in plasma.

The active oxygen species is activated molecules, atoms or ions that are generated in plasma and include oxygen. In order for the oxidation to take place with the amino acids or proteins being inhibited from being damaged, singlet oxygen atoms, excited oxygen molecules or hydroxyl radicals are appropriate for use, and thus, it is preferable for at least one of these to be included.

The active hydrogen is activated molecules, atoms or ions that are generated in plasma and include hydrogen. In order for the reduction to take place with the amino acids or proteins being inhibited from being damaged, excited hydrogen atoms are appropriate for use.

As for the method for generating active oxygen species or active hydrogen, an active oxygen species is generated from water vapor plasma or oxygen plasma, while it is possible to generate active hydrogen from water vapor plasma or hydrogen plasma. In order to generate such types of plasma, a high frequency discharge with a frequency of 1 kHz to 100 MHz or a microwave discharge with 2.5 GHz can be used.

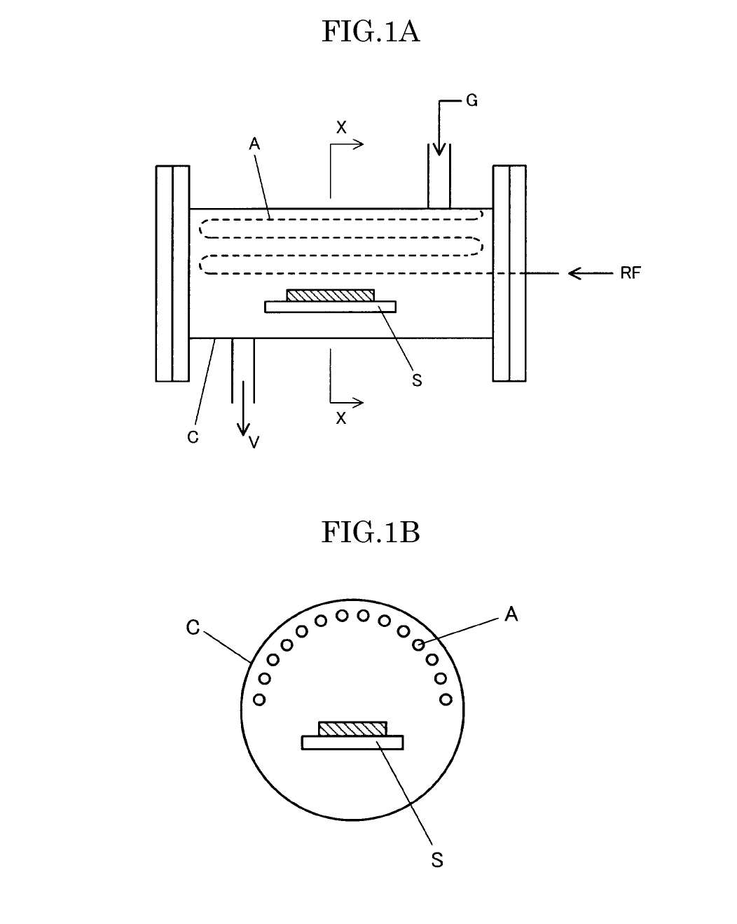

FIGS. 1A and 1B are schematic diagrams showing an example of a plasma-generating device using a high frequency discharge. A vacuum container C made of stainless steel (diameter of 20 cm×length of 45 cm) is provided with an inductively coupled (ICP) antennae A in the proximity of a wall of the vacuum container. The antennae A is used as a capacitively coupled (CCP) antennae, and thus is set so that a high frequency (RF) voltage can be applied and no current can flow.

The plasma-generating device is not limited to that in FIGS. 1A and 1B, and a method for generating plasma by introducing microwaves into a vacuum container using a waveguide instead of the antennae A can be adopted. In addition, a method for releasing plasma into the air as torch plasma, such as atmospheric pressure helium torch plasma, and a method for generating plasma in the air through the below-described dielectric barrier discharge can also be adopted without using a vacuum container. In the case where plasma with high output is used, it is necessary to lower the output as long as the plasma is turned on to such an extent that the amino acids or the proteins are not damaged, and at the same time to take into consideration the distance between the location where plasma is generated and the object to be oxidized or reduced. In particular, it is possible to carry out efficient treatment by using a plasma diffusion region while inhibiting amino acids or proteins from being damaged.

In the plasma-generating device in FIGS. 1A and 1B, the air is expelled from the vacuum container C as indicated by arrow V using a vacuum pump so that the inside is converted to a vacuum state, and then a gas of which the pressure is under control is introduced as indicated by arrow G. The pressure of the gas depends on the type of gas to be introduced, and in the case of water vapor, the pressure is adjusted in a range from several Pa to several hundreds of Pa. The high frequency voltage applied to the antennae A is adjusted depending on the type and the pressure of the gas to be introduced, and a voltage with a frequency of 1 kHz to 100 MHz and with an input power of several tens of W to several hundreds of W is used.

A plasma-generating region and a plasma diffusion region are created in concentric form around the antennae A to which a high frequency voltage is applied. The proximity of the antennae, within a range of 1 cm or less, is filled with high energy electrons, and the energy level of the electrons is high around this range (within a range approximately 5 cm from the antennae), and thus, the electrical field is not cancelled and there is a sheath region where plasma is generated. There is a plasma diffusion region where the energy level of some electrons is mitigated around the sheath region (approximately 5 cm or more away from the antennae). In the case where water vapor is converted to plasma, for example, there is much active hydrogen in the plasma-generating region and there are many hydroxyl radicals in the plasma diffusion region.

FIG. 1B is a cross-sectional diagram along X-X in FIG. 1A. The symbol S is an object to be oxidized or reduced, and amino acids, proteins or an object that includes these are placed on a calcium fluoride substrate, for example. As for the method for placing them, a seed may be placed directly thereon, or amino acids or proteins in powder form may be dissolved in water, applied to the substrate, and dried so as to be used.

A high frequency voltage of 13.56 MHz and 50 W was applied to the antennae A in FIG. 1, water vapor was introduced as the gas G, and the spectrum within the vacuum container was measured in the proximity of the antennae A, which is in the upper portion of the vacuum container in FIG. 1B, in the mid-portion of the vacuum container and in the lower portion of the vacuum container, which was at distance from the antennae A. As a result of the measurement, singlet oxygen atoms with a wavelength of 777 nm, excited oxygen molecules with a wavelength of 762 nm and hydroxyl radicals with a wavelength of 309 nm were observed as active oxygen species. In addition, excited hydrogen atoms with a wavelength of 486 nm and 656 nm were observed as active hydrogen.

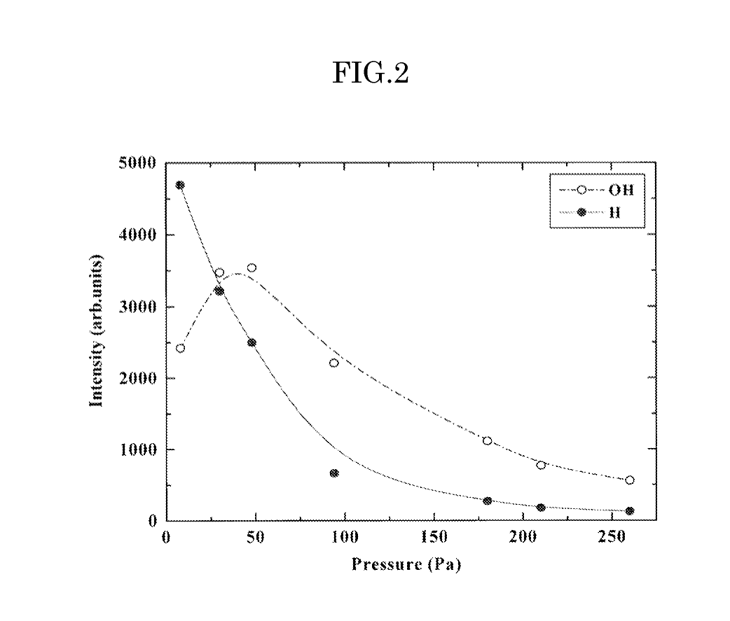

Next, it was confirmed in the case where the water vapor pressure was changed as in FIG. 2 that the amount of the hydroxyl radicals (OH) was greater than the amount of the excited hydrogen atoms (H) for the water vapor pressure of 100 Pa to 150 Pa, and the amount of the excited hydrogen atoms (H) was greater than the amount of the hydroxyl radicals (OH) for the water vapor pressure of 3 Pa to 30 Pa on the basis of the intensity of the spectrum within the vacuum container.

Therefore, it is preferable for the water vapor pressure to be 100 Pa to 150 Pa as the conditions where oxidation due to an active oxygen species can be expected in the case where water vapor plasma is used as the plasma. In the case where oxygen is supplied as the gas, naturally, only an active oxygen species is generated. In the case where oxygen plasma induces oxidation, oxygen ions are easily generated, though depending on the energy level of the plasma, and therefore, it is necessary to note that the risk of amino acids or proteins being destroyed is high.

In addition, it is preferable for the water vapor pressure to be 3 Pa to 30 Pa as the conditions where reduction due to active hydrogen can be expected in the case where water vapor plasma is used. In the case where hydrogen is supplied as the gas, naturally, only active hydrogen is generated.

In the plasma oxidation-reduction method and in the method for promoting plant/animal growth using the same according to the present invention, it is preferable to use an active oxygen species or active hydrogen in the plasma diffusion region that is at a predetermined distance or greater away from the plasma-generating electrode. In the plasma-generating region at a predetermined distance or less away from the electrode, in particular, damage to the amino acids and proteins is significant, and it is difficult to induce oxidation or reduction to these or to effectively promote or inhibit the growth of plants or animals.

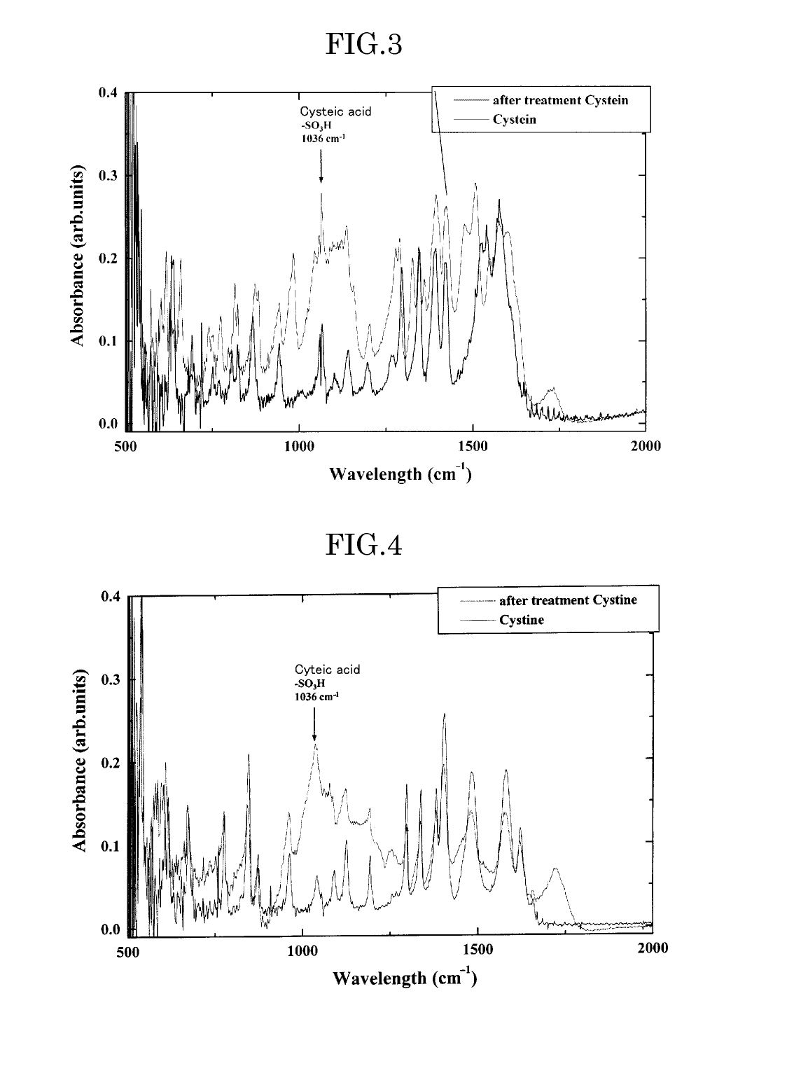

Oxidation or reduction treatment was carried out for 60 minutes on the amino acids, cystein and cystine, which were the object S in FIG. 1. FIG. 3 shows the change in the spectrum before and after treatment on the Fourier transform infrared ray (FTIR) spectrum of cystein. Absorption at the wavelength of 1036 cm⁻¹ (—SO₃H, cysteic acid), which is specific to cystein, was reduced after the treatment, from which it can be inferred that cystein was oxidized to cystine.

FIG. 4 shows a change in the spectrum before and after the treatment of the FTIR spectrum of cystine. Absorption at the wavelength of 1036 cm⁻¹ (—SO₃H), which is specific to cystein, was increased after the treatment, from which it can be inferred that cystine was reduced to cystein.

As a result, it can be seen that the use of the plasma oxidation-reduction method according to the present invention can make the amino acids cystein and cystine oxidized or reduced with high and stable reproducibility.

Cystein and cystine are amino acids within cells, particularly within transcription factors (HSF, Nrf2 and the like), and greatly relate to the state of activation of the transcription factors. The transcription factors are activated by converting the cystein within the transcription factors to cystine, and thus, the process for transcribing the genetic information of the DNA to RNA can be promoted.

In the plasma oxidation-reduction method according to the present invention, as shown in FIGS. 3 and 4, it is possible to directly oxidize or reduce amino acids or proteins in the case where it is possible to make an active oxygen species or active hydrogen make direct contact with these objects.

However, there is moisture around the amino acids or proteins within cells, and therefore, an active oxygen species, such as hydroxyl radicals, converts the moisture to hydrogen peroxide. This hydrogen peroxide works to oxidize the amino acids or proteins within the cells.

In particular, it is possible to convert the moisture within cells to hydrogen peroxide, to make the hydrogen peroxide oxidize cystein in the transcription factors within the cells, and to activate the transcription factors.

It is also possible to convert the moisture within the cells to hydrogen peroxide, to make the hydrogen peroxide oxidatively modify the cystein within the cells (cystein outside the transcription factors) so that cysteic acid is generated, and to make the cysteic acid activate the transcription factors within the cells, for example, accumulate heat shock proteins.

Thus, the transcription factors within cells can be activated so as to make it possible to enhance the glycolytic system within the cells, the TCA circuit or the electron transfer system, and thus to promote the growth of the living body. In addition, it is possible for active hydrogen to work on amino acids or proteins so as to exercise the reducing effects, even in the case where there is moisture. This converts cystine to cystein so that the transcription factors are inhibited from being activated, and as a result, growth of the living body is inhibited.

Next, seeds of daikon radish sprouts (dry state) were used as the object in FIG. 1, and the growth change was observed. A high frequency of 13.56 MHz with a power consumption of 50 W was supplied to the plasma-generating device (using a vacuum container made of stainless steel with a diameter of 20 cm×a length of 45 cm) that was used for plasma irradiation, and an oxygen gas with a pressure of 80 Pa was used so that plasma irradiation treatment was carried out for 60 minutes. In order to compare the effects of the plasma irradiation, seeds of daikon radish sprouts irradiated with plasma and seeds not irradiated with plasma were left to grow for four days in the same environment. Table 1 shows the results. 300 seeds were used for each condition. The numerical values in Table 1 are average values for each condition.

TABLE 1

| | | | |
|---|---|---|---|
| Feature of daikon | Irradiated | Not irradiated | Ratio of |
| radish sprout | with plasma | with plasma | increase |

| | | | | | |
|---|---|---|---|---|---|
| Total length | 167 | mm | 120 | mm | 39.1% |
| Length of stem | 70 | mm | 41 | mm | 70.3% |
| Length of root | 97 | mm | 79 | mm | 22.8% |
| Width of seed leaf | 10.6 | mm | 9.5 | mm | 11.6% |

It can be easily understood from the results in Table 1 that the use of the plasma oxidation-reduction method according to the present invention makes it possible to greatly promote the growth of daikon radish sprouts.

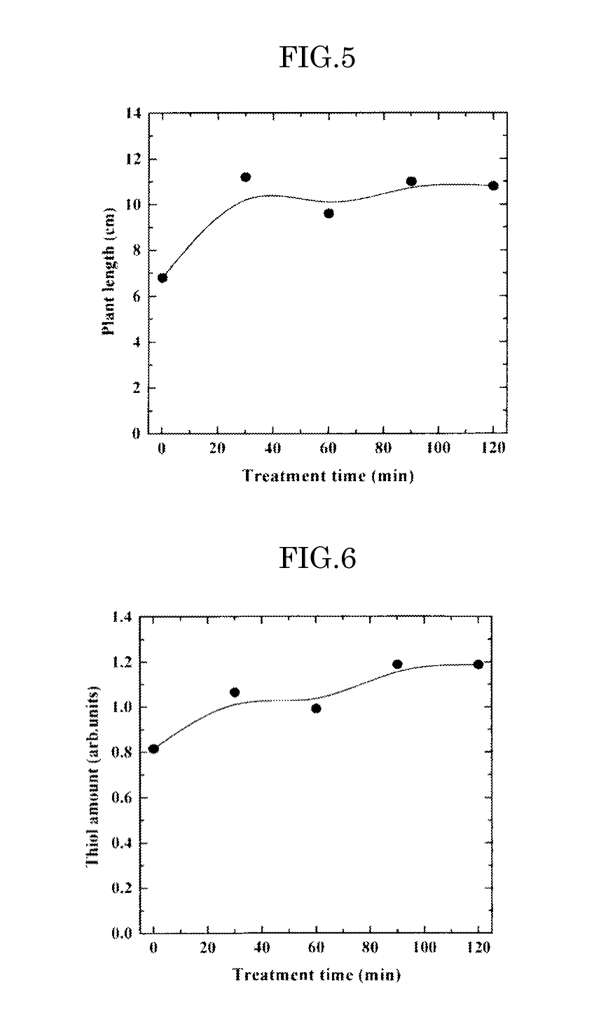

Furthermore, water vapor with a pressure in a range from several Pa to 50 Pa was made to flow into the same plasma-generating device to which a high frequency of 13.56 MHz was applied so that plasma was generated. Seeds of daikon radish sprouts were treated with this plasma for the periods of time in FIGS. 5 and 6. In order to check for a change in the amount of thiol within the seeds due to the plasma treatment, a thiol quantification kit (ANASPEC) and a micro-plate reader (Thermo FCskan) were used.

FIG. 5 shows the dependency of the length of a daikon radish sprout (stem plus root) on the period of time irradiated with plasma. It can be seen that the length of a daikon radish sprout increases as the time of plasma irradiation elapses. FIG. 6 shows a change in the amount of thiol in a seed of a daikon radish sprout as plasma irradiation progresses. The amount of thiol increases depending on the period of time irradiated with plasma due to the reducing effects of the plasma, and it can be seen that the dependency of the amount of thiol on the period of time irradiated with plasma and the change in the length of a daikon radish sprout have the same tendency. This indicates the possibility that the amount of thiol within a seed relates to the growth of a plant.

In the case of plasma treatment with the water vapor pressure varying, the oxidation-reduction properties of amino acids (cystein sample) were evaluated using the peaks that appeared at 2578 cm⁻¹ (thiol group) and at 520 cm⁻¹ (disulfide bond) in the FTIR spectrum.

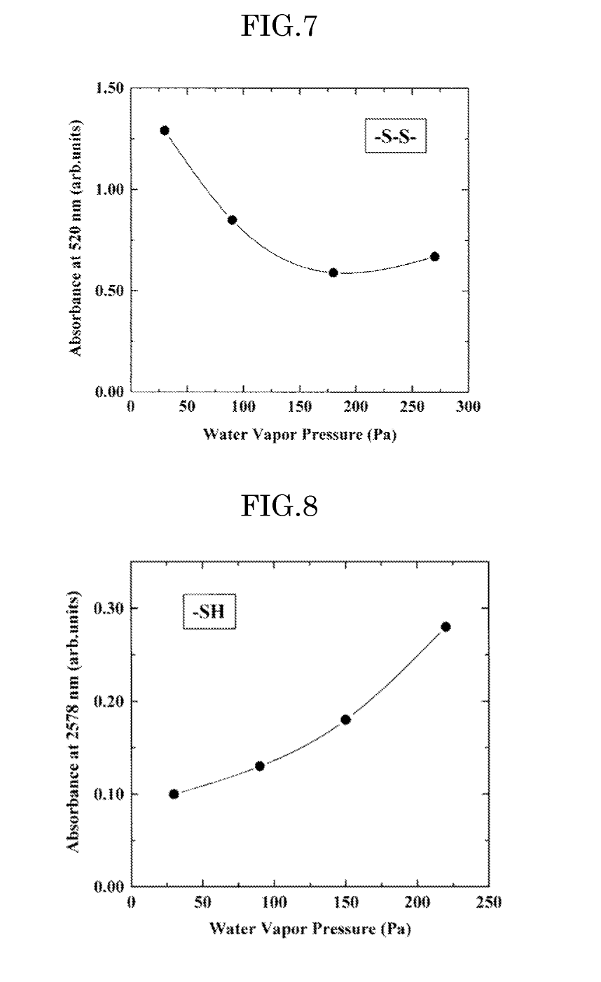

FIG. 7 shows the measurement of a change in the disulfide bond (—S—S—) relative to the water vapor pressure where the absorbance in the spectrum of the disulfide bond increases as the water vapor pressure decreases. This is considered to be because cystine, which has a disulfide bond, increases.

FIG. 8 shows the measurement of a change in the thiol group (—SH) relative to the water vapor pressure where the absorbance of the thiol group decreases as the water vapor pressure decreases. This is considered to be because cystein, which has a thiol group, is reduced and cystine or the cysteic acid gained by oxidatively modifying cystein increases.

It is possible for the cause of the increase in the amount of thiol in FIG. 6 to be the conversion of cystine to cystein due to the reduction by active hydrogen. When only cystein increases, the growth of the living body tends to be inhibited. However, there are many hydroxyl radicals because water vapor is converted to plasma, and thus, there are oxidation effects together with the reduction effects, and therefore, as shown in FIG. 7, cystine is generated or cystein is oxidatively modified to cysteic acid so that the amounts of cystine and cysteic acid both increase to activate the transcription factors within the cells, which is considered to promote growth.

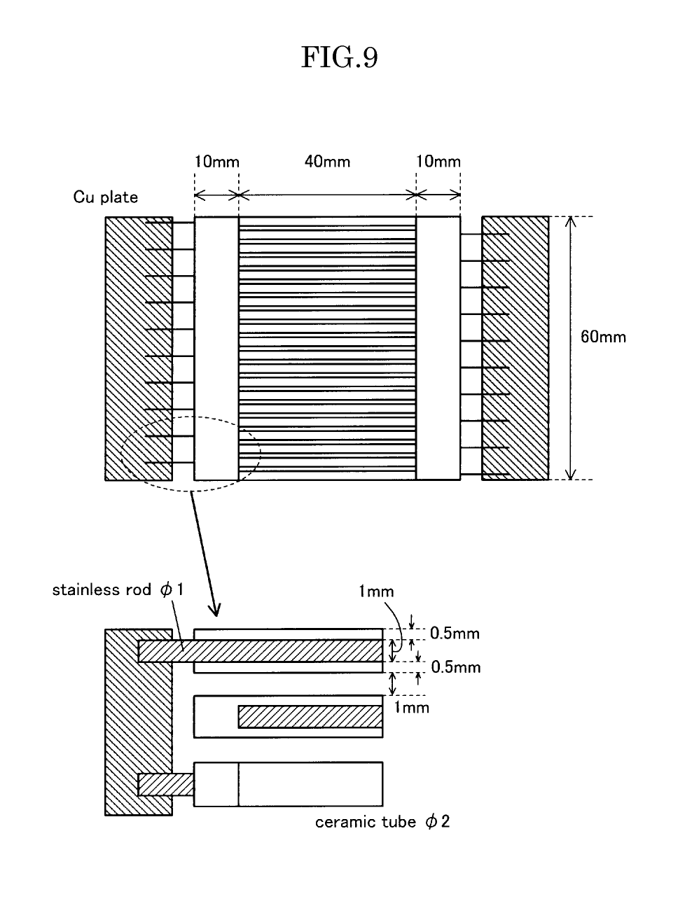

Next, growth change in the budding yeast was checked using the plasma-generating device in FIG. 9. FIG. 9 shows the structure of the electrodes for dielectric barrier discharge, where 20 rods made of stainless steel having a diameter of 1 mm and a length of 60 mm and covered with a ceramic tube with an outer diameter of 2 mm are alternately placed for each electrode. The discharge section is a region where the electrodes are placed so as to be meshed with each other and has a length of 40 mm in the lateral direction in the figure and a length of 60 mm in the longitudinal direction. The distance between the rods is 1 mm as shown in FIG. 9.

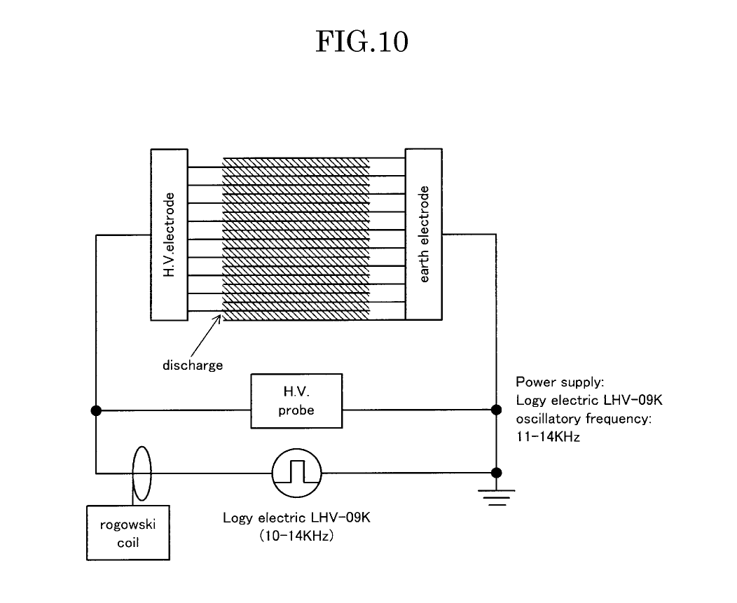

It is possible to change the discharge region of the plasma-generating device in FIG. 10 by adjusting the length and the number of rods in the arrangement, and thus, the discharge region is adjusted depending on the type and the amount of plants or animals to be treated with plasma.

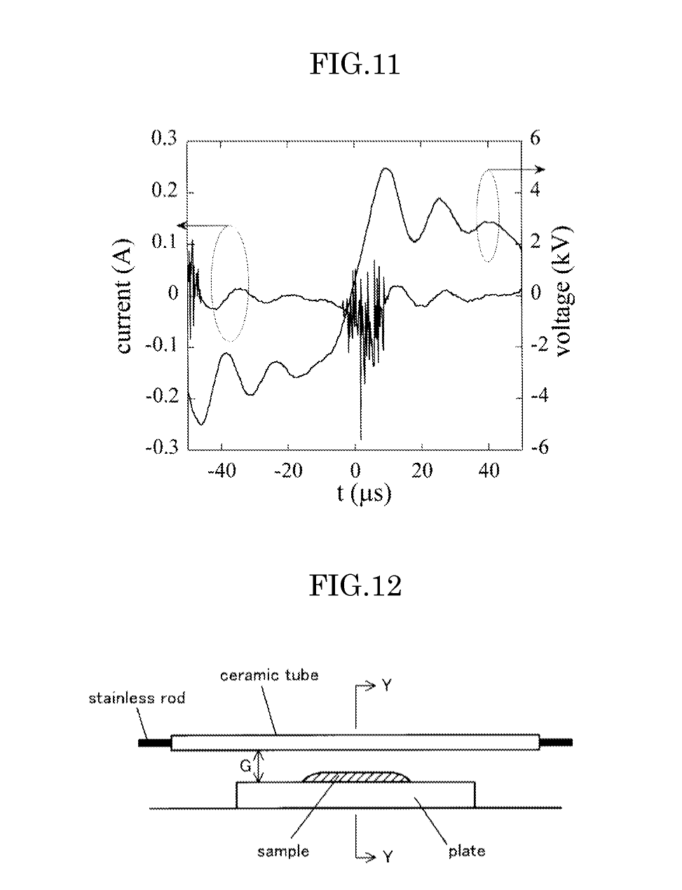

FIG. 10 is a diagram schematically showing the structure of the circuit of the plasma-generating device in FIG. 9. A pulse voltage was supplied to the discharge electrodes in FIG. 9. The power supply used in the experiment was LHV-09K made by Logy Electric Co., Ltd., and the applied voltage had a frequency of 10 kHz and a peak-to-peak voltage Vₚ₋ₚ of 10 kV. The voltage supplied to the discharge electrodes was measured using a high voltage probe, and a supplied current was measured using a Rogowski coil type current probe. FIG. 11 shows the current-voltage wave for one cycle. It can be understood from the graph in FIG. 11 that Vₚ₋ₚ was 10 kV and the peak of the discharge current was 0.11 A.

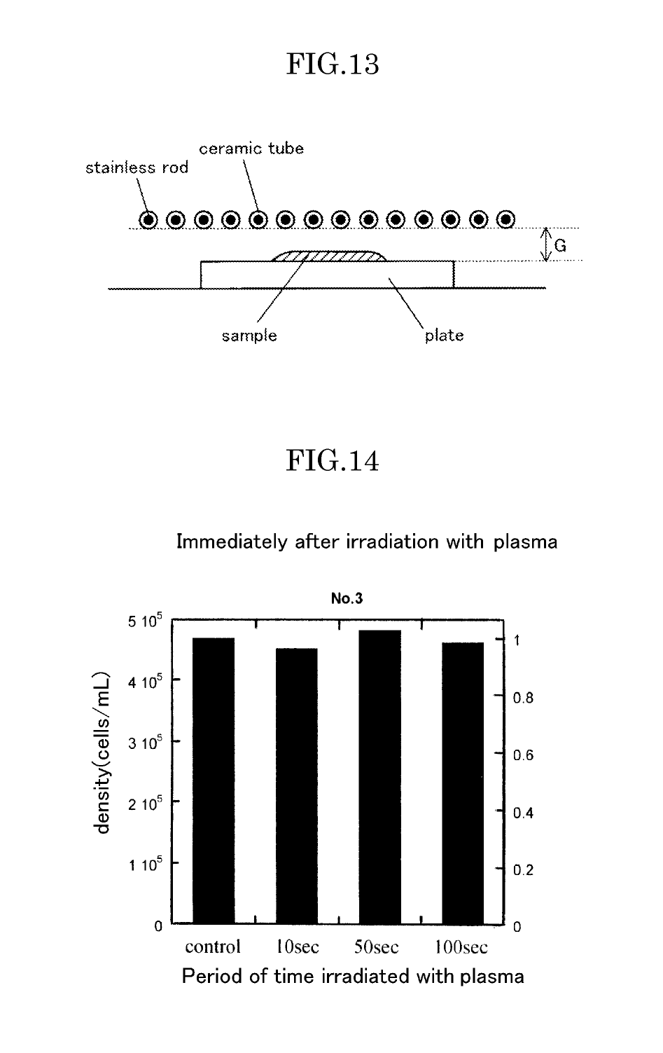

The object, budding yeast, was treated with plasma using the plasma-generating device in FIGS. 9 and 10, and the growth change was observed. As shown in FIG. 12, the electrodes in the plasma-generating device (stainless steel rods covered with a ceramic tube) were placed at a distance G away from a glass substrate, which was the plate for a sample. FIG. 13 is a cross-sectional diagram along arrow Y-Y in FIG. 12. The budding yeast was placed on the glass substrate as the object (sample).

A budding wild strain (BY21391) was used as the budding yeast. A yeast-floating liquid having a yeast concentration shown in FIG. 14 (approximately 4 to 5×10⁵ cells·mL) was prepared, 50 μL of which was placed on the glass substrate (1 cm×1 cm square) as a drop. The discharge electrodes were placed in a location at a distance G of 2 mm from the glass substrate, and plasma irradiation was carried out in the air. In order to evaluate the effects of the plasma irradiation, the objects not irradiated with plasma and the objects irradiated for a 10-second period of time, a 50-second period of time and a 100-second period of time were checked. The objects treated with plasma (or not treated with plasma) were put into a tube with the glass substrate, mixed with a culture medium of 0.95 mL so as to be 1 mL, and cultivated. As for the cultivation method, shaking culture was used, and the concentration of the yeast was measured using a cell counting board.

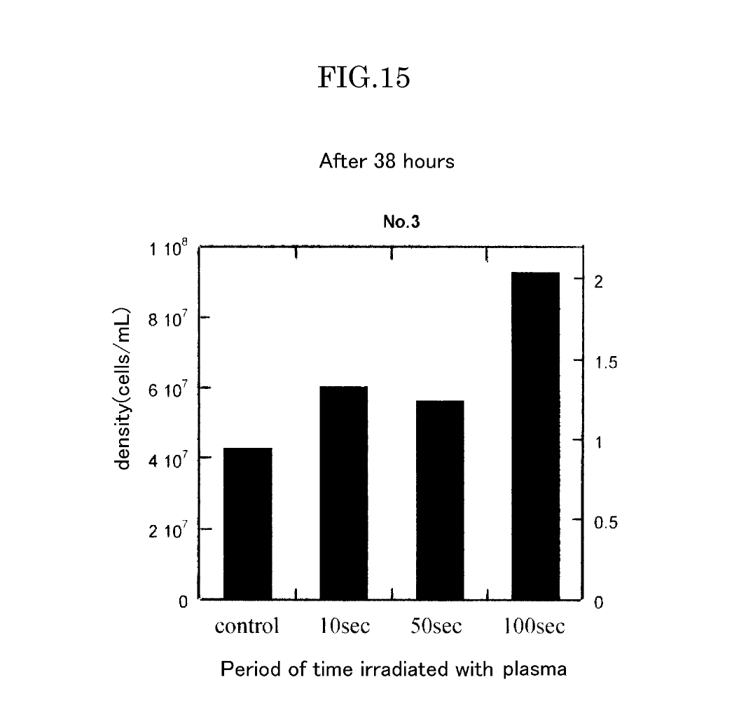

The graph in FIG. 14 shows the state of the budding yeast immediately after being irradiated with air plasma in atmospheric pressure, and the graph in FIG. 15 shows the state of the budding yeast after 38 hours had elapsed since being irradiated with plasma. In all the cases of the budding yeast being irradiated with plasma, an increase in the number of colonies (two times at maximum) was observed in comparison with the case of the budding yeast not irradiated with plasma.

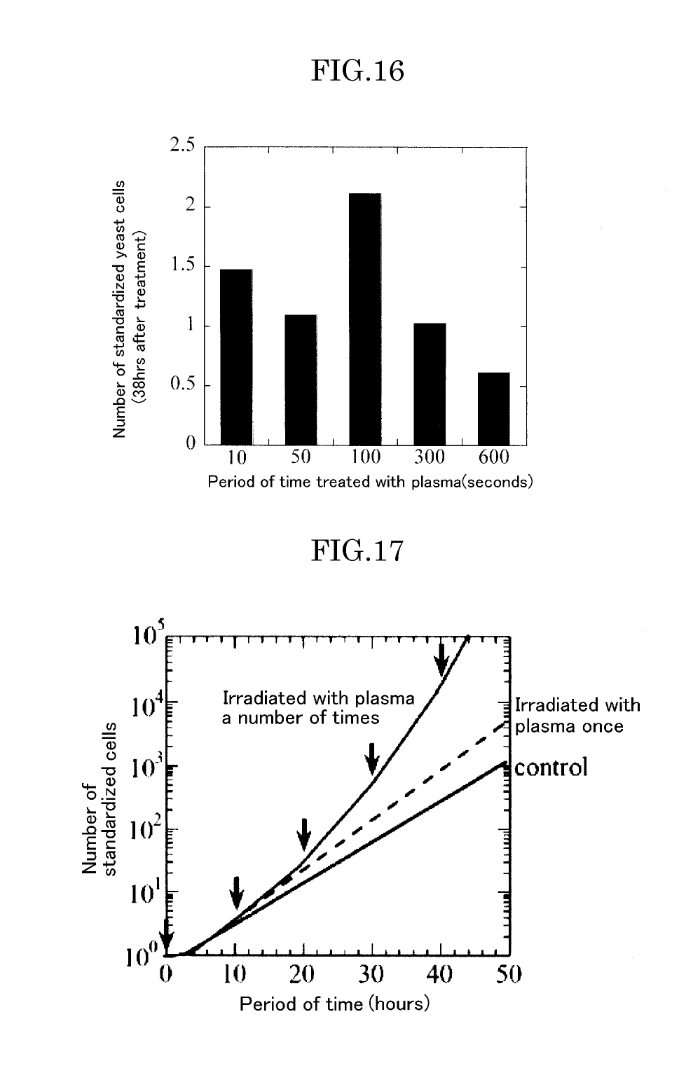

Furthermore, the period of time of irradiation was set to 10 seconds, 50 seconds, 100 seconds, 300 seconds and 600 seconds in order to check any changes in the promotion of the growth relative to the period of time of irradiation with plasma. The state after 38 hours of cultivation was evaluated using the number of yeasts in the case where the budding yeast not irradiated with plasma was standardized as 1. FIG. 16 shows the results.

As the results in FIG. 16 show, acceleration in the proliferation of the yeast was observed for the irradiation up to 300 seconds. In particular, it was found that the acceleration in the proliferation of the yeast has a maximum value for the irradiation close to 100 seconds (in a range from 50 seconds to 300 seconds).

Furthermore, in order to evaluate the effects of plasma irradiation in the case where plasma irradiation is carried out a number of times, plasma irradiation was carried out five times in total for every 10 hours since the initial plasma irradiation. As shown in the graph in FIG. 17, in the case where plasma irradiation is carried out a number of times, it was confirmed that the effects of acceleration in the proliferation are greater as compared to the case where plasma irradiation is carried out once.

It can be understood that the plasma oxidation-reduction method according to the present invention effectively works on cystein or cystine within the transcription factors as described above, and in addition to that, other effects can be inferred, for example, the ions and radicals in the plasma accelerate the process for generating acetyl-CoA from sugars, fatty acids and amino acids (glycolytic system), and the environment is converted so that the CoA catalyst can be easily activated (pH of cytoplasm). It is also assumed that acetyl-CoA is oxidized in the TCP circuit to H₂O and CO₂, ions and radicals work on the process for generating NADH and ATP, the pH within the cells is changed to reinforce the catalytic action of the enzymes, and the enzymes themselves within the cells change. It is also possible that cyclin and cyclin-dependent kinase, which are proteins for controlling the cell cycle, are oxidized or reduced so that the activities of these are changed to accelerate or inhibit the cell cycle.

The dielectric barrier discharge takes place in the air between the electrodes in FIG. 13 (between ceramic tubes). Therefore, the distance G between the electrodes and the object in FIGS. 12 and 13 has great effects in order for the active oxygen species or active hydrogen generated in the plasma to be efficiently supplied to the object. Though the figures show the distance vis-à-vis the plate for a sample, the distance vis-à-vis the object changes depending on the form of the plate and the size of the object, and therefore, the distance G between the electrode and the object to be treated is used for the description.

In the case where the distance G is smaller than 1 mm, the object is affected by the plasma generated between the electrodes, which makes it easy for the amino acids or the proteins in the plant or the animal to be damaged, which is not preferable. Meanwhile, the distance over which the active oxygen species or active hydrogen reaches in the air is affected by various conditions, including the atmospheric pressure and the movement of the air. However, it has been confirmed in the case where the distance G is 15 mm that the effects of plasma oxidation-reduction that are necessary in the method for promoting plant/animal growth seldom appear. Therefore, it is preferable for the distance G to be set in a range from 1 mm to 15 mm.

The size of the object to be treated, which is a plant or an animal, is not particularly limited as long as only the surface of the object is treated. In the case where the entirety is treated simultaneously, however, it is preferable for the size to be set to 5 mm or less.

### INDUSTRIAL APPLICABILITY

As described above, according to the present invention, amino acids or proteins that form a living body are controlled using plasma, and in particular, amino acids or proteins are oxidized or reduced using plasma, and thus, it is possible to provide a plasma oxidation-reduction method with which it is possible to control the structure of the amino acids or the proteins with high and stable reproducibility. In addition, it is possible to provide a method for promoting plant/animal growth by using this plasma oxidation-reduction method. Furthermore, it is possible to provide a plasma-generating device for use in this method for promoting plant/animal growth.

### EXPLANATION OF SYMBOLS

A antennae
C vacuum container
G gas for plasma
S object

1. A plasma oxidation-reduction method, comprising the step of oxidizing or reducing amino acids or proteins by using an active oxygen species in plasma or active hydrogen in plasma.

2. The plasma oxidation-reduction method according to claim 1, wherein the active oxygen species comprises any one of singlet oxygen atoms, excited oxygen molecules, or hydroxyl radicals, and the active hydrogen comprises excited hydrogen atoms.

3. The plasma oxidation-reduction method according to claim 1, wherein the active oxygen species is generated from water vapor plasma or oxygen plasma, and the active hydrogen is generated from water vapor plasma or hydrogen plasma.

4. The plasma oxidation-reduction method according to claim 1, wherein the active oxygen species or the active hydrogen is generated through high frequency discharge or microwave discharge.

5. The plasma oxidation-reduction method according to claim 1, wherein water vapor plasma is used as the plasma, and oxidation occurs due to the active oxygen species when the water vapor pressure is in a range from 100 Pa to 150 Pa.

6. The plasma oxidation-reduction method according to claim 1, wherein water vapor plasma is used as the plasma, and reduction occurs due to the active hydrogen when the water vapor pressure is in a range from 3 Pa to 30 Pa.

7. The plasma oxidation-reduction method according to claim 1, wherein an active oxygen species or active hydrogen in a plasma diffusion region is used.

8. The plasma oxidation-reduction method according to claim 1, wherein the amino acids are within cells.

9. The plasma oxidation-reduction method according to claim 1, wherein the active oxygen species or the active hydrogen directly oxidizes or reduces the amino acids or the proteins.

10. The plasma oxidation-reduction method according to claim 1, wherein there is moisture around the amino acids or the proteins, the active oxygen species converts the moisture into hydrogen peroxide, and the hydrogen peroxide oxidizes the amino acids or the proteins.

11. The plasma oxidation-reduction method according to claim 1, wherein the amino acids are within transcription factors.

12. The plasma oxidation-reduction method according to claim 1, wherein the amino acids are cystein or cystine.

13. The plasma oxidation-reduction method according to claim 12, wherein the active oxygen species oxidizes cystein.

14. The plasma oxidation-reduction method according to claim 12, wherein the active hydrogen reduces cystine.

15. The plasma oxidation-reduction method according to claim 8, wherein an object with cells having moisture is irradiated with the active oxygen species so that the moisture is converted to hydrogen peroxide and the hydrogen peroxide oxidizes cystein in the transcription factors within the cells, and the transcription factors are activated.

16. The plasma oxidation-reduction method according to claim 8, wherein an object with cells having moisture is irradiated with the active oxygen species so that the moisture is converted to hydrogen peroxide and the hydrogen peroxide oxidatively modifies cystein within the cells so as to generate cysteic acids, and the cysteic acids activate the transcription factors within the cells.

17. A method for promoting plant/animal growth using the plasma oxidation-reduction method according to claim 1.

18. The method for promoting plant/animal growth according to claim 17, wherein an object irradiated with the active oxygen species or the active hydrogen is plant or animal cells.

19. The method for promoting plant/animal growth according to claim 18, wherein the plant or animal cells are of a seed, the plant or animal cells are contained in a vacuum container, and the plant or animal cells are irradiated with an active oxygen species or active hydrogen through high frequency discharge or microwave discharge.

20. The method for promoting plant/animal growth according to claim 18, wherein the plant or animal cells have a size of 5 mm or less, and the plant or animal cells are placed in the air at a distance of 1 mm to 15 mm away from the electrode for dielectric barrier discharge, and the plant or animal cells are irradiated with the active oxygen species or the active hydrogen.

21. The method for promoting plant/animal growth according to claim 20, wherein the plant or animal cells are dispersed in a liquid.

22. A plasma-generating device for use in a method for promoting plant/animal growth, comprising: a vacuum container; an inductively coupled antennae placed in the proximity of a wall of the container; and a means for placing plant or animal cells in a region where the plasma generated by the antennae diffuses.

23. A plasma-generating device for use in a method for promoting plant/animal growth, comprising a pair of electrodes for dielectric barrier discharge, each of which is made of a number of metal rods coated with a ceramic tube, that are meshed with each other so that the metal rods are parallel to each other, wherein the electrodes and plant or animal cells are placed in the air at a distance of 1 mm to 15 mm away from each other.
